(19) United States
(12) Patent Application Publication
(10) Pub. No.: US 2026/0007384 A1
(43) Pub. Date: Jan. 8, 2026

(54) SYSTEMS AND METHODS FOR MONITORING AND TREATMENT OF INJURIES IN A PATIENT POPULATION USING ONE OR MORE WEARABLE DEVICES

(71) Applicant: THE JOHNS HOPKINS UNIVERSITY, Baltimore, MD (US)

(72) Inventors: Amir MANBACHI, Baltimore, MD (US); Nicholas THEODORE, Baltimore, MD (US); Aliaksei PUSTAVOITAU, Baltimore, MD (US)

(21) Appl. No.: 18/993,637

(22) PCT Filed: Jul. 18, 2023
(86) PCT No.: PCT/US2023/027962

§ 371 Date: Jan. 13, 2025

Related U.S. Application Data
(60) Provisional application No. 63/390,711, filed on Jul. 20, 2022.

(51) Int. Cl. A61B 8/06; A61B 8/00

(52) U.S. Cl. CPC A61B 8/06; A61B 8/4227; A61B 8/4494; A61B 8/5223

(57) ABSTRACT

A system includes a plurality of wearable ultrasound devices configured to be in contact with a plurality of patients. Each of the wearable ultrasound devices is used to measure one or more parameters of the patients. The system also includes a computing system in communication with the wearable ultrasound devices. The computing system is configured to receive the parameters from the wearable ultrasound devices, determine a plurality of diagnostic states of the patients based at least partially upon the parameters, and determine prioritization of the patients for triage based at least partially upon the diagnostic states.

### CROSS-REFERENCE TO RELATED APPLICATIONS

This application is the national stage entry of International Patent Application No. PCT/US2023/027962, filed on Jul. 18, 2023, and published as WO 2024/019993 A1 on Jan. 25, 2024, which claims the benefit of U.S. Provisional Patent Application No. 63/390,711, filed on Jul. 20, 2022, which are hereby incorporated by reference in their entireties.

### GOVERNMENT SUPPORT

This invention was made with Government support under N66001-20-2-4075, awarded by Department of the Navy. The Government has certain rights in the invention.

### FIELD OF THE DISCLOSURE

The present disclosure relates generally to systems and methods for monitoring and treating injuries. More particularly, the present disclosure relates to systems and methods for determining patient prioritization for triage using a plurality of wearable ultrasound devices.

### BACKGROUND

Mass casualty events in the United States are presently triaged using methods such as the simple triage and rapid treatment (START) system. This system involves first responders categorizing patients into four color categories based on the extent of the patient's injuries. More particularly, the START categorization is based on the following algorithm: if a patient is able to walk, they are given a green tag. If a patient has no respiration after the airway is opened, they are given a black tag. If a patient has a respiratory rate over 30, no radial pulse, and is unresponsive to simple instructions by first responders, they are given a red tag. If the patient does not fall into any of the above categories, they are given a yellow tag. As these current triaging methods rely on few quantitative measurements, systems and methods for improving triaging efficacy and patient outcomes is desired.

### SUMMARY

A system is disclosed. The system includes a plurality of wearable ultrasound devices configured to be in contact with a plurality of patients. Each of the wearable ultrasound devices is used to measure one or more parameters of the patients. The system also includes a computing system in communication with the wearable ultrasound devices. The computing system is configured to receive the parameters from the wearable ultrasound devices, determine a plurality of diagnostic states of the patients based at least partially upon the parameters, and determine prioritization of the patients for triage based at least partially upon the diagnostic states.

A system for determining prioritization of a plurality of patients for triage is also disclosed. The system includes a plurality of wearable ultrasound devices configured to be in contact with the plurality of patients. The patients are simultaneously sick or injured. The patients include at least a first patient and a second patient. The wearable ultrasound devices include one or more first wearable ultrasound devices configured to be in contact with the first patient and one or more second wearable ultrasound devices configured to be in contact the second patient. Each of the wearable ultrasound devices is or includes an adhesive patch. Each of the wearable ultrasound devices includes a flexible transducer element array that includes a plurality of transducer elements. The transducer elements are configured to generate one or more ultrasound acoustic beams that are configured to be electronically steered. The ultrasound acoustic beams are used to measure parameters including temperature, blood flow, blood pressure, blood volume status, sources of bleeding, organ size, organ function, or a combination thereof. The system also includes a computing system in communication with the wearable ultrasound devices. The computing system is configured to receive the parameters from the wearable ultrasound devices, determine a plurality of diagnostic states of the patients based at least partially upon the parameters, and continuously determine and update prioritization of the patients for triage based at least partially upon the diagnostic states. The system also includes a display configured to show the diagnostic states and the prioritization of the patients for triage.

A system for continuously determining and updating prioritization of a plurality of patients for triage is also disclosed. The system includes a plurality of wearable ultrasound devices configured to be in contact with the patients. The patients are simultaneously sick or injured. The patients comprise at least a first patient and a second patient. The wearable ultrasound devices include one or more first wearable ultrasound devices configured to be in contact with the first patient and one or more second wearable ultrasound devices configured to be in contact the second patient. The one or more first wearable ultrasound devices include first and second wearable ultrasound devices configured to monitor a pneumothorax of the first patient, a third wearable ultrasound device configured to monitor a myocardial function, a blood volume status, and a tamponade of the first patient, fourth and fifth wearable ultrasound devices configured to monitor a hemothorax and a pneumoperitoneum of the first patient, and a sixth wearable ultrasound device configured to monitor bladder filling and a hemoperitoneum of the first patient. Each of the wearable ultrasound devices is or includes an adhesive patch. Each of the wearable ultrasound devices includes a flexible transducer element array that includes a plurality of transducer elements. The transducer elements are configured to generate one or more ultrasound acoustic beams that are configured to be electronically steered. The ultrasound acoustic beams are used to measure parameters including temperature, blood flow, blood pressure, blood volume status, sources of bleeding, organ size, and organ function. The blood volume status includes sizes of cardiac chambers and inferior vena cava, a flow across a left ventricular outflow tract, and changes over time with respirations and with fluid or blood administration. The system also includes a computing system in communication with the wearable ultrasound devices. The computing system is configured to receive the parameters from the wearable ultrasound devices. The computing system is also configured to determine a plurality of diagnostic states of the patients based at least partially upon the parameters. The diagnostic states include a location and severity of bleeding, a pulmonary function, a location and an amount of fluid in an abdomen, a tissue perfusion, a cardiac function. The cardiac function is also determined based at least partially upon right and left ventricular filling pressures, regional ventricular wall thickening, and arterial venous blood flow. The diagnostic states also include a hepatic function. The hepatic function is also determined based at least partially upon liver stiffness that is measured using 2D shear-wave elastography. The diagnostic states also include a peripheral function. The peripheral function is also determined based at least partially upon capillary density and tissue oxygenation. The diagnostic states also include a response to medical intervention. The computing system is also configured to generate a 3D reconstruction of one or more portions of the patients based at least partially upon the parameters. The one or more portions include a heart, an airway, and solid abdominal organs. The computing system is also configured to continuously determine and update prioritization of the patients for triage based at least partially upon the diagnostic states and the 3D reconstruction of the patients. The system also includes a display configured to show the diagnostic states, the 3D reconstruction, and the prioritization of the patients for triage.

### BRIEF DESCRIPTION OF THE FIGURES

FIG. 1 illustrates a schematic view of a system for monitoring one or more patients that includes one or more wearable ultrasound devices, according to an embodiment.

FIG. 2 illustrates a perspective view of a transducer element array that may be used in the wearable ultrasound device, according to an embodiment.

FIGS. 3A and 3B illustrate the different views of the transducer element array, according to an embodiment.

FIGS. 4A-4C illustrate schematic views of electronic beam steering by the wearable ultrasound devices, according to an embodiment.

FIGS. 5A and 5B illustrate an example of a closed-loop network analyzer for analyzing data captured by the wearable ultrasound devices, according to an embodiment.

FIG. 6 illustrates a system for determining a range of settings where accurate flow measurements can be acquired, according to an embodiment.

FIGS. 7A-7I illustrate mass casualty triaging using the system, according to an embodiment.

### DETAILED DESCRIPTION

The system and method described herein may determine patient prioritization for triage during mass trauma events. More particularly, the system and method may continuously monitor patient parameters to provide the ability to continuously determine patient prioritization over time, which may improve the speed, quantity, and efficacy of life-saving interventions. The system and method may include artificial intelligence (AI) functionality to maintain an optimal imaging window (e.g., maintain view of relevant structures in an image), perform pattern recognition for diagnostic applications, provide historical records of the same tissue for interpretable measurement comparisons, perform data integration with 3D reconstruction, or a combination thereof.

The system and method may include one or more wearable ultrasound wearable devices (e.g., patches) that are configured to measure one or more parameters of a patient. For example, a plurality patients may each have a plurality of wearable ultrasound devices attached thereto simultaneously. The wearable ultrasound devices may include a flexible transducer, which may have a variable geometry for ultrasound beam formation and the simultaneous use of multiple probes. The ultrasound acoustic beam may be electronically steered via on-site operators, remote steering, or by an automated system based on artificial intelligence (AI) functionality.

The parameters that may be measured by the wearable ultrasound devices may include temperature, organ size, organ function, blood flow, blood pressure, blood volume status, sources of bleeding, or a combination thereof. The blood volume status assessment may include the size of the cardiac chambers and inferior vena cava, the flow across the left ventricular outflow tract, and their changes over time with respirations and with fluid or blood administration. In one embodiment, the system and method may determine diagnostic states such as the severity of the bleeding and/or the patient's response to intervention based upon the parameters. In another embodiment, the system and method may determine diagnostic states such as pulmonary function, cardiac function, fluid in the abdomen, flow in the renal arteries and in the lower extremities based upon the parameters. In yet another embodiment, the system and method may determine diagnostic states such as anatomical structures (e.g., B-mode) and tissue perfusion (e.g., Doppler) based upon the parameters. In yet another embodiment, the system and method may perform anatomic evaluation and/or 3D reconstruction of the heart, airway, and/or solid abdominal organs based upon the parameters. The ability to perform anatomic evaluation and 3D reconstruction using continuous ultrasound supplants the need for ionizing radiation, making the procedure safer for both patient and clinician.

Uses of this technology may include cardiac, pulmonary, and abdominal evaluation and monitoring of trauma victims while transporting via emergency medical services, with image evaluation performed by the receiving center. This technology may also or instead be used for trauma surgery, anesthesia, critical care, military operations, flight physiology, and care in austere environments. Critical care uses may additionally include monitoring of mechanical ventilation therapy (e.g., lung recruitment, ventilator weaning).

pulmonary, and abdominal evaluation and monitoring of wounded combat victims during evacuation with image evaluation performed en route to or by the receiving center. Flight physiology uses may include venous flow during long-distance flight. In addition, this technology may be used to monitor surgical patients intraoperatively, evaluate surgical patients at risk for acute renal failure, monitor surgical and critically ill patients for deep vein thrombosis, assess airway anatomy, and support airway management. Furthermore, the development of remote, non-contact patient vital sensing with smart phones or tablets expands access of important medical information to high-density patient settings, as well as for patients who may have limited access to traditional in-person care.

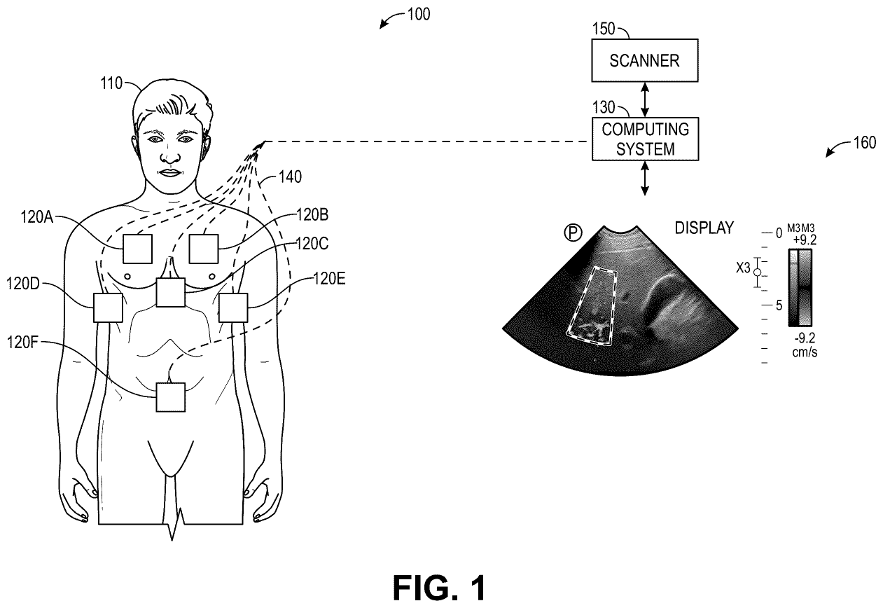

FIG. 1 illustrates a schematic view of a system 100 for monitoring one or more patients (one patient is shown: 110), according to an embodiment. The system 100 may include a plurality of wearable ultrasound devices (six are shown: 120A-120F) that may be in contact with (e.g., attached to) the patient 110. The wearable ultrasound devices 120A-120F may be configured to remain in contact with the patient without being held in place by a person. The wearable ultrasound devices 120A-120F may be or include patches, bands (e.g., wristbands, armbands, belts, etc.), clothing, or the like.

The wearable ultrasound devices 120A-120F may be designed with higher operational frequencies to analyze the microvasculature of the patient, which can be used as a surrogate for diagnosing shock. In the particular example shown, the devices 120A, 120B may be configured to monitor the pneumothorax; the device 120C may be configured to monitor myocardial function, blood volume status, and/or tamponade; the devices 120D, 120E may be configured to monitor the hemothorax and/or pneumoperitoneum; and the device 120F may be configured to monitor bladder filling and/or hemoperitoneum. In an embodiment, the wearable ultrasound devices 120A-120F may be structurally the same, but programmed differently based upon the location on the patient 100 where they are placed, as illustrated in Table 1 below, which shows the different probe geometries, frequencies, and depths.

The system 100 may also include a (e.g., wireless) global positioning system (GPS) device, which may be attached to the patient 110. In one embodiment, the GPS device may be part of (e.g., attached to or positioned within) one or more of the wearable ultrasound devices 120A-120F. The GPS device may provide the patient's location to medical professionals (e.g., first responders) upon a critical health status change.

The system 100 may also include a computing system 130. In one embodiment, the wearable ultrasound devices 120A-120F may be connected to the computing system 130 via one or more wires 140. In another embodiment, the system 100 may include a wireless receiver (e.g., a handheld scanner) 150. When placed within a predetermined distance (e.g., 10 cm or less) from the wearable ultrasound devices 120A-120F, the scanner 150 may communicate with (e.g., receive data from) the wearable ultrasound devices 120A-120F. The scanner 150 may then transmit the data to the computing system 130. Alternatively, the scanner 150 may be part of the computing system 130. For example, they may both be part of a handheld device such as a tablet or cell phone. The computing system 130 may process the (e.g., ultrasound) data from the wearable ultrasound devices 120A-120F. The computing system 130 may also prioritize patient treatment based upon the processed data, as described in greater detail below.

The computing system 130 may include or be part of an external base station, which may facilitate bi-directional, Wi-Fi-based communication between the wearable ultrasound devices 120A-120F and PC-based data acquisition software running on the computing system 130. Recording may be wirelessly configured 8/12-bit at up to 50 kHz total sampling. The programmable constant current stimulation circuit may have an analog front end capable of filtering, amplifying, and conditioning up to 6 (or more) biosignals and wireless powering via inductive coupling from a 13.56 MHz external primary power source during recording. The software on the computing system 130 may allow for user-configurable settings for stimulation parameters, sampling frequency, sampling resolution, dynamic calibration, and recharging. The computing system 130 may be capable of the dynamic, wireless recording of ECG/slow waves, temperature, motion, respiration, and neural activity. As a result of hardware-based conditioning, the computing system 130 may require minimal post-processing of data, negating the need for techniques commonly required in other studies (e.g., down sampling).

The system 100 may also include a display 160 that is configured to show the processed ultrasound data (e.g., as images). The display 160 may continuously output B-mode, color Doppler, power Doppler, tissue Doppler echocardiography, calculated flows, structure sizes, or a combination thereof. The display 160 may also show the current order of priority of treatment (e.g., treat patient A before treating patient B). In one embodiment, the computing system 130, scanner 150, and/or display 160 may all be part of the same handheld device.

Table 1 details transducer specifications and the corresponding assessments that may be used to triage trauma patients using the wearable ultrasound devices 120A-120F. In addition to the examples provided in Table 1, cardiac function may also be monitored by measuring the right and left ventricular filling pressures and the regional ventricular wall thickening, as well as monitoring the arterial and venous blood flow. Hepatic function may be assessed by monitoring liver stiffness using 2D real-time shear-wave elastography, and peripheral function may be estimated using analyses of microcirculation, including capillary density and photoacoustic imaging for tissue oxygenation.

TABLE 1

| Monitoring | | Probe | Probe | Imaging | |
|---|---|---|---|---|---|
| (function) | Indication | geometry | frequency | depth | Assessments |
| Pneumothorax | Trauma | Linear, phased, | 3-12 MHz | 5-6 cm | Lung sliding: |
| (pulmonary) | | convex | | | presence/absence |
| | | | | | Lung pulse: |
| | | | | | presence/absence |
| | | | | | B-lines: |
| | | | | | presence/absence |
| Extravascular lung | Fluid | Linear, phased, | 3-8 MHz | 10-12 cm | B-lines: |
| fluid (pulmonary) | therapy | convex | | | presence/absence |
| LVOT (CV) | SV/CO | Phased | 1-5 MHz | 15-20 cm | Flow across LVOT |
| Size of RV and | SV/CO | Phased | 1-5 MHz | 15-20 cm | Size of the RV and |
| LV (CV) | | | | | LV |
| LVEF (CV) | LV systolic | Phased | 1-5 MHz | 15-20 cm | EF: change in size of |
| | function | | | | LV between systole |
| | assessment | | | | and diastole |
| Size of IVC (CV) | Volume | Phased | 1-5 MHz | 15-20 cm | Size of IVC |
| | status | | | | Variability of IVC |
| | assessment | | | | |

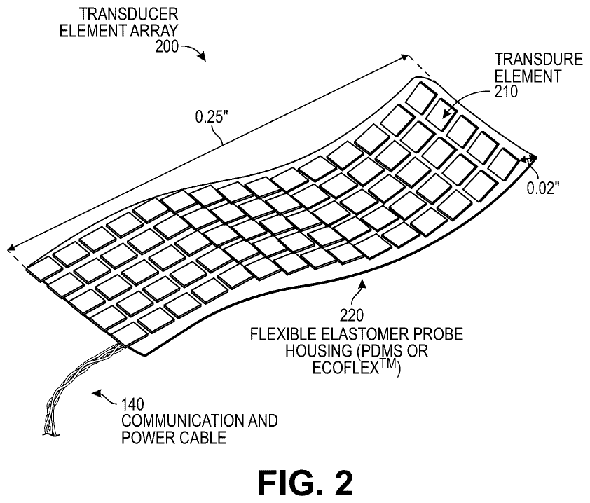

Each of the wearable ultrasound devices 120A-120F may include a transducer element array, according to an embodiment. FIG. 2 illustrates a perspective view of a transducer element array 200, according to an embodiment. The transducer element array 200 may be a 2D or 3D array that includes a plurality of elements 210. The transducer elements 210 may also or instead be referred to as probes. The transducer elements 210 may be or include capacitive micromachined ultrasonic transducer (CMUT) elements or piezoelectric elements. The transducer elements 210 may be combined in any arbitrary shape (e.g., square, rectangle, circle, etc.) and housed on or in a flexible (e.g., elastomer) membrane 220. Soft workable elastomers such as polydimethylsiloxane (PDMS) or EcoFlex™ may be used to make molds of any shape to house the transducer elements 210. The stiffness of these materials is adjustable in the casting process (E˜50-500 kPa), and they can match the shape near the skin of the patient 110 to allow a good acoustic contact between the probe and the target (e.g., the heart), eliminating or mitigating the need for gels. They can be cast into versatile re-usable modular (e.g., CMUT or piezoelectric) array ‘patches’ that may be used individually for sensing or combined together in a frame to form a larger probe. Alternatively, for field applications (e.g., military), they may cast-customized for every subject, allowing uninterrupted contact with a fixed region of the skin, enabling continuous monitoring.

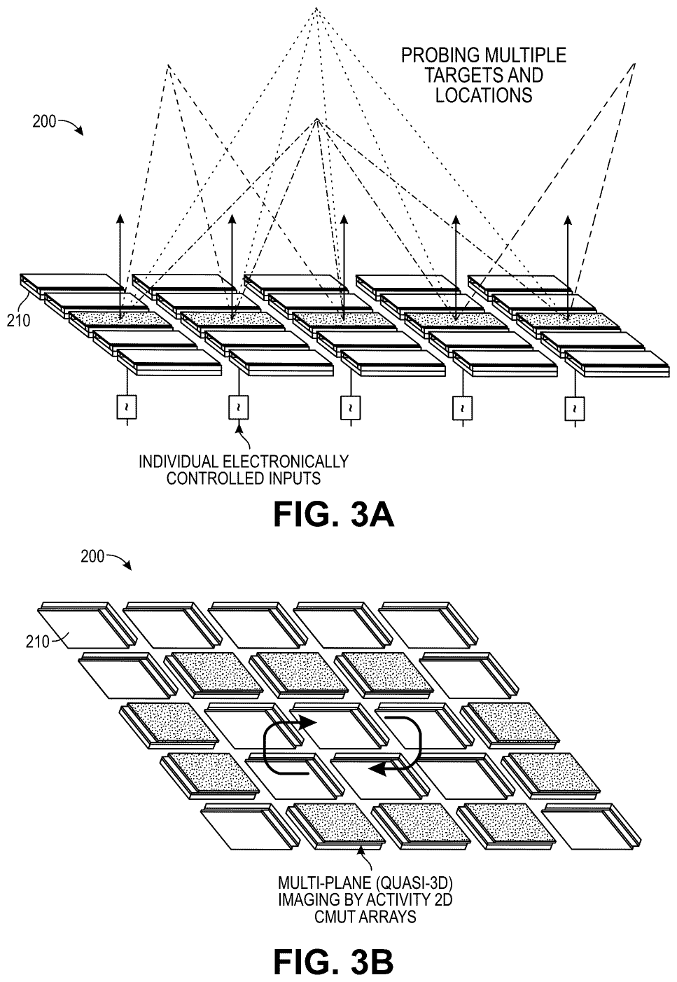

FIGS. 3A and 3B illustrate the different views of the transducer element array 200, according to an embodiment. The transducer elements 210 may allow a variety of beam adjustments. As shown in FIG. 3A, curved and/or linear wave-fronts may be obtained by varying the timing and/or excitation signal of the transducer elements 210. As the transducer elements 210 are capable of generating a range of frequencies, the probe can focus on targets at various depths optimally. The independent nature of the transducer elements 210 allows for grouping of any number of transducer elements 210 to form multiple probes on-the-go. For example, the same set of transducer elements 210 may all be used to scan one target (e.g., the heart), or can be sub-divided into multiple probes and scan several targets (e.g., the heart and lungs) simultaneously. Several targets may be scanned simultaneously with a single transducer element array 200 without scanning (e.g., focusing on) the volume between the targets. As shown in FIG. 3B, the 2D arrangement of transducer elements 210 in the flexible housing allows for multiple views of the same target (e.g., the heart) to be scanned by different transducer elements 210 at the same time, allowing for tomographic reconstruction revealing 3-dimensional characteristics.

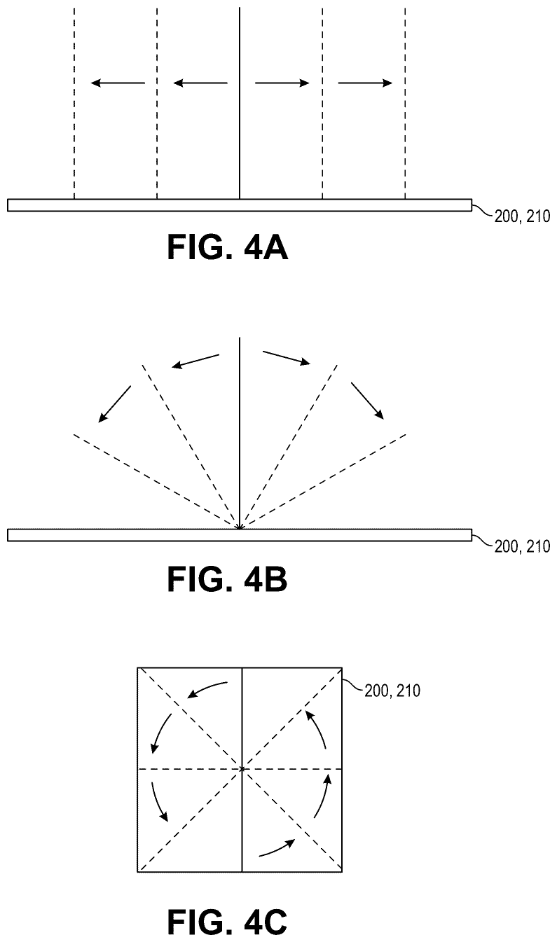

FIGS. 4A-4C illustrate schematic views of electronic beam steering by the wearable ultrasound devices 120A-120F, according to an embodiment. More particularly, FIGS. 4A-4C illustrate electronic beam steering by the transducer element array 200 (or the transducer element 210). To allow optimal imaging without direct adjustments of the transducer element arrays 200 (or the transducer elements 210), the beams formed may be steered in 3-dimensional space from their original positions (solid line) to additional positions (dashed lines). Only some additional beam positions are demonstrated along the entire spectrum of possible positions. More particularly, FIG. 4A illustrates a side view of the transducer element array 200 (or the transducer element 210) showing that the original beam can be moved along the length of the transducer element array 200 (or the transducer element 210). FIG. 4B illustrates a side view of the transducer element array 200 (or the transducer element 210) showing that the formed beam can be tilted at an angle to the probe. FIG. 4C illustrates a bottom view of the transducer element array 200 (or the transducer element 210) showing that the formed beam can be rotated around its central axis.

To ensure the success of the fabrication processes, the characterization approach may be twofold: closed- and open-system verification.

### Closed-System Verification

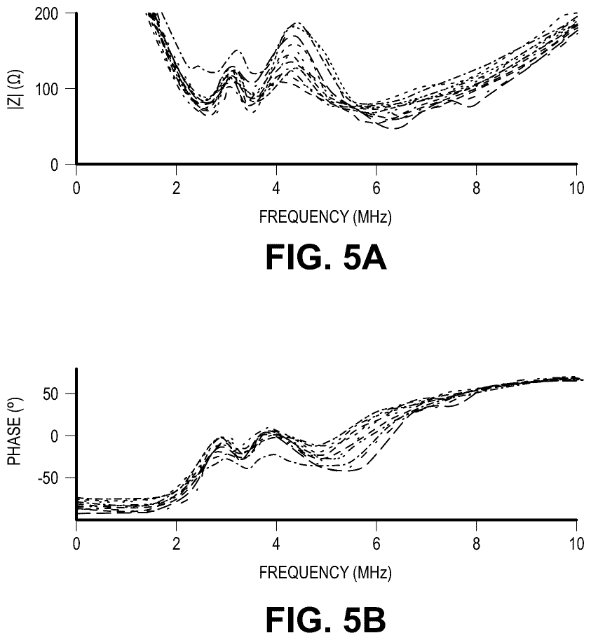

Each fabricated transducer may be attached to a network analyzer to study the peaks and valleys (i.e., resonance and anti-resonance frequencies) and to ensure that the center frequency and the operating frequency range (i.e., the −3 dB, FWHM) align with the intended design. FIGS. 5A and 5B illustrate an example of a closed-loop network analyzer characterization from the preliminary data captured by the system 100. More particularly, FIG. 5A illustrates sample impedance, and FIG. 5B illustrates phase results obtained from a network analyzer demonstrating the resonance and antiresonance frequencies of the transducer array 200.

### Open-System Verification

The acoustic pressure profile may be tested and verified to ensure the focal depth and beam width align with the intended design. This may be achieved using a water tank and several hydrophones in various frequency ranges.

The data captured by the wearable ultrasound devices 120A-120F may be noisy, potentially due to the EMI generated by the pump and the ultrasound system. Hardware (e.g., copper coating on internal electronics) and software (e.g., bandpass filter) solutions may help reduce noise. Lowering the power or duty cycle, as well as different materials for encapsulation of the sensor can reduce overheating of the wearable ultrasound devices 120A-120F. Furthermore, a temperature sensor that automatically shuts the wearable ultrasound devices 120A-120F off when it reaches a temperature threshold may be incorporated into the devices 120A-120F to maintain patient safety. The wearable ultrasound devices 120A-120F may use focused transmit beamforming or plane-wave ultrasound. Plane-wave ultrasound images require fewer scanlines, which reduces the burden of device memory and time. Using plane waves may result in decreased image quality. However, decreased image quality may not have a negative impact, as changes in the blood flow and changes of state (e.g., absence of blood in the abdomen to presence of blood in the abdomen; or absence of pneumothorax to presence of pneumothorax) may still be detected.

### Fabricated Device Functionality

To assess the functionality of fabricated transducer array 200, a CIRS multi-purpose, multi-tissue ultrasound device may be used to measure the axial and lateral resolution of the system 100, depth penetration, and ensure accurate contrast measurements for cysts of varying echogenicity. Acceptable contrast measurements may be determined to be within ±3 dB of the contrast measured using an ultrasound system for anechoic, hypoechoic, and hyperechoic cysts and a wire located at 3 cm in depth. Acceptable resolution may be determined to be less than 2 mm axial and lateral resolution.

### Heating Profile

The heating profile from the wearable ultrasound devices 120A-120F may be measured by covering the face of the transducer array 200 in gel and placing a thermocouple between the transducer face and a tissue mimicking phantom. The time that the wearable ultrasound devices 120A-120F can continuously acquire doppler images before reaching a predetermined temperature threshold (e.g., 111° F.) may be used to define the duration where imaging can be conducted safely. Flow quantification accuracy

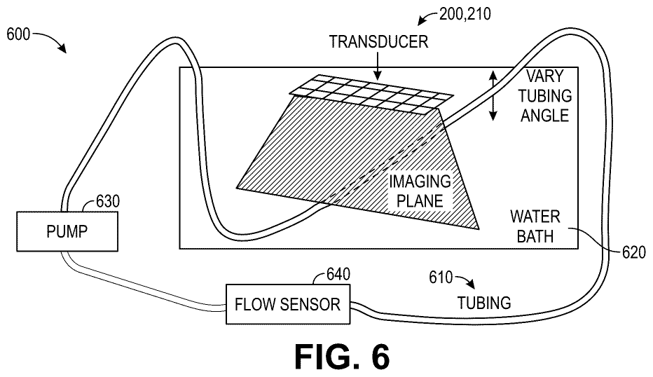

FIG. 6 illustrates a system 600 for determining a range of settings where accurate flow measurements can be acquired, according to an embodiment. The system 600 may include a (e.g., 12 mm diameter) tubing 610 immersed in a water bath 620 and at the focus of the transducer array 200 (or transducer element 210). A peristaltic pump 630 may be used to control the flow through the tubing 610, and a flow rate monitor 640 may be used to acquire reference flow rate values. The angle of the tubing 610 relative to the transducer array 200 (or transducer element 210) and the speed of fluid through the tubing 610 may be varied from 1° to 60° and 6-18 cm/s, respectively. To minimize the heat generated by the transducer array 200 (or transducer element 210), the duty cycle of the transducer array 200 (or transducer element 210) pulses may be varied from 20% to 100%. At each flow rate, angle, and duty cycle combination, the percent error of the Doppler measurements from the reference may be calculated and compared to the flow rate error from a conventional transducer.

Once an initial round of data has been acquired for the patient 110, information such as the locations of bones, soft tissues, organs, etc. may be registered by the system 100 (or 600). This may allow for the subsequent scans to be optimized for the patient/organ-specific purpose, significantly reducing the scanning time. For example, the presence of bones may impact ultrasound images because of the high-density gradients at the interfaces. If the locations of the bones in relationship to the probe are known a priori, then they may be avoided in subsequent imaging attempts by electronically steering the beam away or using alternative transducer elements 210 to scan the same regions.

Second, by monitoring organ function continuously for long time periods, the system 100 (or 600) may be able to search for patterns resembling specific pathological/physiological states enabling timely prediction (potentially life- and organ-saving).

Third, the system 100 (or 600) can integrate information from multiple transducer element arrays 200 that perform scans of the same organ in multiple planes, allowing a 3D tomographic reconstruction to be performed. The coordinate transformation matrix from the first pass may be used to be generate real-time 3D ultrasound images of the organs.

The system 100 (or 600) may also compare/contrast data received from different patients that may be subject to similar (e.g., military) field conditions. This can help in making effective pre-emptive clinical decisions.

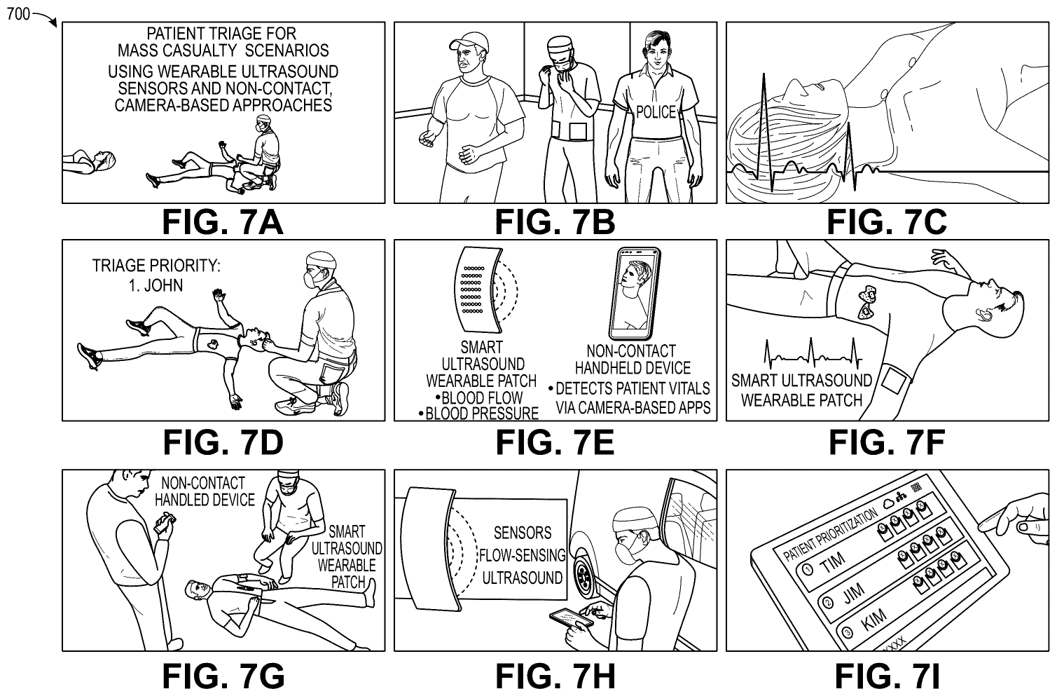

FIGS. 7A-7I illustrate mass casualty triaging using the system 100, according to an embodiment. Upon arrival to a mass casualty scene (FIG. 7A), it may be difficult to determine which patients should be prioritized. FIG. 7B shows how stress may lead to inefficient triaging. FIG. 7C shows that patients may have internal and/or or external injuries that are critical for determining the patient's status. FIG. 7D shows scanning the wearable ultrasound devices 120A-120F (FIG. 7E) with the non-contact handheld scanning device 150 to determine the parameters (FIG. 7F). FIG. 7G shows non-contact scanning (e.g., remote photoplethysmography) using the handheld scanning device 150 to provide additional information. FIG. 7H shows the ultrasound devices 120A-120F providing the parameters (e.g., vital signs) in addition to flow-sensing ultrasound, which allows for prioritization and identification of patients in a safe, effective manner, as shown in FIG. 7I.

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.” The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

1. A system, comprising:
a plurality of wearable ultrasound devices configured to be in contact with a plurality of patients, wherein each of the wearable ultrasound devices is used to measure one or more parameters of the patients; and
a computing system in communication with the wearable ultrasound devices, wherein the computing system is configured to:
receive the parameters from the wearable ultrasound devices;
determine a plurality of diagnostic states of the patients based at least partially upon the parameters; and
determine prioritization of the patients for triage based at least partially upon the diagnostic states.

2. The system of claim 1, wherein the patients comprise at least a first patient and a second patient, and wherein the wearable ultrasound devices comprise one or more first wearable ultrasound devices configured to be in contact with the first patient and one or more second wearable ultrasound devices configured to be in contact the second patient.

3. The system of claim 1, wherein each of the wearable ultrasound devices comprises a patch including a flexible transducer element array that has a plurality of transducer elements, wherein the transducer elements are configured to generate one or more ultrasound acoustic beams that are configured to be electronically steered by the computing system, and wherein the ultrasound acoustic beams are used to measure the parameters.

4. The system of claim 1, wherein the parameters comprise temperature, blood flow, blood pressure, blood volume status, sources of bleeding, organ size, organ function, or a combination thereof.

5. The system of claim 1, wherein a first of the wearable ultrasound devices is configured to monitor a pneumothorax of a first patient of the patients.

6. The system of claim 5, wherein a second of the wearable ultrasound devices is configured to monitor a myocardial function, a blood volume status, a tamponade, or a combination thereof of the first patient.

7. The system of claim 6, wherein a third of the wearable ultrasound devices is configured to monitor a hemothorax, a pneumoperitoneum, or both of the first patient.

8. The system of claim 7, wherein a fourth of the wearable ultrasound devices is configured to monitor bladder filling, a hemoperitoneum, or both of the first patient.

9. The system of claim 1, further comprising:
a scanner that is configured to wirelessly scan the wearable ultrasound sensors to receive the parameters therefrom, wherein the computing system receives the parameters from the scanner; and
a display configured to show the diagnostic states and the prioritization of the patients for triage.

10. The system of claim 9, wherein the scanner, the computing system, and the display are part of a single handheld device.

11. A system for determining prioritization of a plurality of patients for triage, the system comprising:
a plurality of wearable ultrasound devices configured to be in contact with the plurality of patients,
wherein the patients are simultaneously sick or injured,
wherein the patients comprise at least a first patient and a second patient,
wherein the wearable ultrasound devices comprise one or more first wearable ultrasound devices configured to be in contact with the first patient and one or more second wearable ultrasound devices configured to be in contact the second patient,
wherein each of the wearable ultrasound devices comprises an adhesive patch,
wherein each of the wearable ultrasound devices comprises a flexible transducer element array that includes a plurality of transducer elements,
wherein the transducer elements are configured to generate one or more ultrasound acoustic beams that are configured to be electronically steered, and
wherein the ultrasound acoustic beams are used to measure parameters including temperature, blood flow, blood pressure, blood volume status, sources of bleeding, organ size, organ function, or a combination thereof,
a computing system in communication with the wearable ultrasound devices, wherein the computing system is configured to:
receive the parameters from the wearable ultrasound devices;
determine a plurality of diagnostic states of the patients based at least partially upon the parameters; and
continuously determine and update prioritization of the patients for triage based at least partially upon the diagnostic states; and
a display configured to show the diagnostic states and the prioritization of the patients for triage.

12. The system of claim 11, wherein the one or more first wearable ultrasound devices comprises:
first and second wearable ultrasound devices configured to monitor a pneumothorax of the first patient,
a third wearable ultrasound device configured to monitor a myocardial function, a blood volume status, and a tamponade of the first patient,
fourth and fifth wearable ultrasound devices configured to monitor a hemothorax and a pneumoperitoneum of the first patient, and
a sixth wearable ultrasound device configured to monitor bladder filling and a hemoperitoneum of the first patient.

13. The system of claim 11, wherein the blood volume status comprises sizes of cardiac chambers and inferior vena cava, a flow across a left ventricular outflow tract, and changes over time with respirations and with fluid or blood administration.

14. The system of claim 11, wherein the diagnostic states comprise:
a location and severity of bleeding;
a pulmonary function;
a location and an amount of fluid in an abdomen;
a tissue perfusion;
a cardiac function;
a response to medical intervention; or
a combination thereof.

15. The system of claim 11, wherein the computing system is also configured to generate a 3D reconstruction of one or more portions of the patients based at least partially upon the parameters, wherein the one or more portions comprise a heart, an airway, solid abdominal organs, or a combination thereof, and wherein the display is configured to show the 3D reconstruction.

16. A system for continuously determining and updating prioritization of a plurality of patients for triage, the system comprising:
a plurality of wearable ultrasound devices configured to be in contact with the patients,
wherein the patients are simultaneously sick or injured,
wherein the patients comprise at least a first patient and a second patient,
wherein the wearable ultrasound devices comprise one or more first wearable ultrasound devices configured to be in contact with the first patient and one or more second wearable ultrasound devices configured to be in contact the second patient,
wherein the one or more first wearable ultrasound devices comprises:
first and second wearable ultrasound devices configured to monitor a pneumothorax of the first patient,
a third wearable ultrasound device configured to monitor a myocardial function, a blood volume status, and a tamponade of the first patient,
fourth and fifth wearable ultrasound devices configured to monitor a hemothorax and a pneumoperitoneum of the first patient, and
a sixth wearable ultrasound device configured to monitor bladder filling and a hemoperitoneum of the first patient,
wherein each of the wearable ultrasound devices comprises an adhesive patch,
wherein each of the wearable ultrasound devices comprises a flexible transducer element array that includes a plurality of transducer elements,
wherein the transducer elements are configured to generate one or more ultrasound acoustic beams that are configured to be electronically steered,
wherein the ultrasound acoustic beams are used to measure parameters including temperature, blood flow, blood pressure, blood volume status, sources of bleeding, organ size, and organ function, and
wherein the blood volume status comprises sizes of cardiac chambers and inferior vena cava, a flow across a left ventricular outflow tract, and changes over time with respirations and with fluid or blood administration;
a computing system in communication with the wearable ultrasound devices, wherein the computing system is configured to:
receive the parameters from the wearable ultrasound devices;
determine a plurality of diagnostic states of the patients based at least partially upon the parameters, wherein the diagnostic states comprise:
a location and severity of bleeding;
a pulmonary function;
a location and an amount of fluid in an abdomen;
a tissue perfusion;
a cardiac function, wherein the cardiac function is also determined based at least partially upon right and left ventricular filling pressures, regional ventricular wall thickening, and arterial venous blood flow;
a hepatic function, wherein the hepatic function is also determined based at least partially upon liver stiffness that is measured using 2D shear-wave elastography;
a peripheral function, wherein the peripheral function is also determined based at least partially upon capillary density and tissue oxygenation; and
a response to medical intervention;
generate a 3D reconstruction of one or more portions of the patients based at least partially upon the parameters, wherein the one or more portions comprise a heart, an airway, and solid abdominal organs; and
continuously determine and update prioritization of the patients for triage based at least partially upon the diagnostic states and the 3D reconstruction of the patients; and
a display configured to show the diagnostic states, the 3D reconstruction, and the prioritization of the patients for triage.

17. The system of claim 16, further comprising a scanner that is configured to wirelessly scan the wearable ultrasound sensors to receive the parameters therefrom, wherein the computing system receives the parameters from the scanner.

18. The system of claim 17, wherein the scanner, the computing system, and the display are part of the same handheld device.

19. The system of claim 16, wherein the wearable ultrasound devices each comprise a global positioning system (GPS) to identify locations of the patients.

20. The system of claim 16, wherein the wearable ultrasound devices each include a temperature sensor that is configured to measure a temperature, and wherein the wearable ultrasound devices are configured to turn off in response to the temperature exceeding a predetermined threshold to prevent heat from the wearable ultrasound from burning the patients.
